(19) United States
(12) Patent Application Publication
(10) Pub. No.: US 2024/0252355 A1
(43) Pub. Date: Aug. 1, 2024

(54) METHOD FOR PROVIDING CONTROL DATA FOR AN OPHTHALMOLOGICAL LASER OF A TREATMENT APPARATUS FOR CORRECTING PRESBYOPIA

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/427,087

(22) Filed: Jan. 30, 2024

(30) Foreign Application Priority Data
Feb. 1, 2023 (DE) 10 2023 102 467.1

(51) Int. Cl. A61F 9/008; G16H 20/40

(52) U.S. Cl. CPC A61F 9/00808; A61F 9/00827; G16H 20/40; A61F 2009/00878

(57) ABSTRACT

The invention relates to a method for providing control data for an ophthalmological laser of a treatment apparatus for correcting presbyopia, wherein the method includes, as steps, ascertaining first correction data for an eye of a patient for correcting the presbyopia from predetermined visual disorder data; ascertaining second correction data for the other eye of the patient, wherein the second correction data is calculated by means of a calculation operation of the first correction data with a patient-specific parameter; and providing the control data for the ophthalmological laser, which includes the first and second correction data for the respective eyes.

### FIELD

The invention relates to a method for providing control data for an ophthalmological laser of a treatment apparatus for correcting presbyopia. In addition, the invention relates to a treatment apparatus with at least one eye surgical laser and at least one control device for performing the method, to a computer program and to computer-readable medium.

### BACKGROUND

Treatment apparatuses and methods for controlling ophthalmological lasers for correcting an optical visual disorder and/or pathologically and/or unnaturally altered areas of the cornea are known in the prior art. Therein, pulsed lasers and a beam focusing device can for example be formed such that laser pulses effect a photodisruption and/or photoablation in a focus situated within the organic tissue to remove a tissue, in particular a tissue lenticule, from the cornea.

Presbyopia can also be treated by means of such methods in that a refractive power of the cornea is changed, wherein multifocal areas in the manner of varifocal glasses can for example also be generated in the cornea. Thus, areas for a near zone and/or a far zone can for example be generated in the cornea.

It is the object of the present invention to provide control data for an improved presbyopia correction.

This object is solved by the independent claims. Advantageous embodiments of the invention are disclosed in the dependent claims, the following description as well as the figures.

### SUMMARY

The invention is based on the idea that both eyes of a patient, which were separately considered and treated heretofore, are correlated with each other in the treatment planning for a presbyopia correction. This means that the treatment of one eye has effects on the treatment planning of the other eye, whereby better treatment results can be achieved.

An aspect of the invention relates to a method for providing control data for an ophthalmological laser of a treatment apparatus for correcting presbyopia, wherein the method comprises the following steps performed by a control device. Therein, an appliance or an appliance component, in particular a processor or microprocessor, is meant by a control device, which is formed for performing the following steps.

In the method, ascertaining first correction data for an eye of a patient for correcting the presbyopia from predetermined visual disorder data, ascertaining second correction data for the other eye of the patient, wherein the second correction data is calculated by means of a calculation operation of the first correction data with a patient-specific parameter, and providing the control data for the ophthalmological laser, which includes the first and second correction data for the respective eyes, are effected.

In other words, a target refraction for both eyes is related or correlated, and after ascertaining the correction for one eye, that for the other eye is automatically determined. Hereto, first correction data can first be determined from previously measured visual disorder data. Thus, the first correction data can describe the correction of the presbyopia of the first eye, thus specify, in which areas or zones of the cornea which refractive power change is to be performed. In particular, the correction data can provide a bi-aspherical or multi-aspherical correction, which means that multiple areas in the cornea can be planned for different visual ranges. Ascertaining this first correction data can be performed according to usual methods for the eye of the patient, preferably for a dominant eye of the patient.

For the other or second eye, second correction data can then be determined, which is set depending on the first correction data. Hereto, the first correction data can be correlated with a patient-specific parameter by means of a calculation operation, wherein the calculation operation is a mathematical rule. Preferably, the calculation operation is a basic arithmetic operation, thus multiplying or dividing the first correction data by the patient-specific parameter or adding or subtracting with the patient-specific parameter. In other words, the second correction data for example results from multiplication of the first correction data by the patient-specific parameter.

Therein, the patient-specific parameter is a value, which can be individually preset or predetermined for each patient, in particular based on a situation of the patient. Thus, the patient-specific parameter can for example depend on an age and/or a profession of the patient. Alternatively or additionally, a preoperative refraction and/or a residual accommodation can also be taken into account, by which the patient-specific parameter can be determined.

For example, a multifocality can be planned in the first eye and the same multifocality can for example be inverted or halved in the second eye due to the patient-specific parameter. By inverted, it is meant that in the first eye, a center of the cornea is for example optimized for a near range and a zone adjoining thereto for a far range, and in the second eye, the center of the cornea can be optimized for the far range and the zone adjoining thereto for the near range by inversion. Thus, an eye can for example also obtain a cylinder correction of −1 diopter at 0 degrees and the other eye of −1 diopter at 90 degrees. Preferably, respective transition zones can also be planned for the correction data. The correction data is preferably based on zone corrections, wherein they can also be based on Zernike polynomials, Fourier series or point corrections.

Finally, the thus ascertained correction data can be provided as control data for the ophthalmological laser, which can be controlled by the control data.

By the invention, the advantage arises that a suitable correlation between the two eyes of a patient can be established, whereby a presbyopia correction can be planned in improved manner and a patient satisfaction can be increased.

The invention also includes embodiments, by which additional advantages arise.

In an embodiment the first and/or second correction data includes bi-aspherical, tri-aspherical and/or multi-aspherical refractive power distributions. Preferably, a continuous refractive power distribution, that is a type of infinitesimally aspherical distribution, can also be provided. In other words, multifocal areas can be planned in the cornea, wherein a respective consistent adaptation of the corresponding areas can be adjusted in the other eye by the patient-specific parameter.

In a further embodiment the calculation operation is a multiplication and the patient-specific parameter has a value in a range of values from −1 to 1. Herein, the value of 0 is preferably excluded from the range of values. Therein, a value of −1 means an inversion of the correction data for the other eye and the value of 1 means the use of the same correction data for both eyes. The values of 0.5 and −0.5 are then correspondingly halving of the respective effect. Hereby, suitable ranges of values can be provided for the patient-specific parameter.

In a further embodiment the calculation operation is an addition and the patient-specific parameter has a diopter value in a range of values from −2 diopters to 2 diopters. Therein, the diopter value can be provided for a spherical correction, a defocus correction and/or a cylinder correction. In other words, a deviation between the two eyes of the patient can be preset such that it is not greater than +/−2 diopters.

In a further embodiment a center of the treatment is differently planned for each eye. In particular, the center for a respective eye can be provided between a pupil center and a corneal vertex viewed in radial direction, wherein this center can be different for each eye. Thus, the center of the treatment of the first eye can for example be centered with the pupil center viewed in radial direction and the center of the treatment of the second eye with the corneal vertex viewed in radial direction. Alternatively, an identical center can also be provided for both eyes. By selection of a different center for the respective eye, aberrations can for example be compensated for, in particular coma and/or trefoil.

In a further embodiment the patient-specific parameter is determined based on a preoperative refraction, a residual accommodation, an age and/or a profession. Thus, for young patients, a low patient-specific parameter in a range from 0 to 1 can for example be provided such that the same treatment or a mitigated treatment is performed on both eyes. In contrast, an inversion of the effect for the other eye can be provided on older patients. For example, a profession can also be taken into account, wherein for professions, in which a far vision is more important, an inversion of the effect can be provided for the second eye, thus a factor of less than 0, and for professions, in which a near vision is more important, an identical treatment can be provided for the second eye, preferably with a mitigated effect, thus a factor of greater than 0. A residual accommodation can also be taken into account, wherein the patient-specific parameter can be reduced towards 0 depending on the still available residual accommodation. In other words, a lower effect can be provided for the second eye in case of a high residual accommodation. Hereby, the advantage arises that suitable criteria can be provided for the patient-specific parameter.

In a further embodiment higher order aberrations are also taken into account in the respective correction data. For example, the higher order aberrations can be determined from wavefront measurements, which are then compensated for in the correction data for the respective eye. Target desires for aberrations can also be taken into account, which can be preset by a patient.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by means of optical breakthrough, in particular at least partially separate it by means of photodisruption and/or to ablate corneal layers by means of (photo)ablation and/or to effect a laser induced refractive index change in the cornea and/or the eye lens and/or to increase a crosslinking of the cornea.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In further advantageous configurations of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment;

FIG. 2 depicts a flow diagram of a method according to an exemplary embodiment.

In the figures, identical or functionally identical elements are provided with the same reference characters.

### DETAILED DESCRIPTION

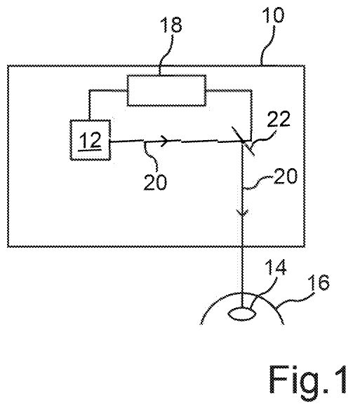

FIG. 1 shows a schematic representation of a treatment apparatus 10 with an ophthalmological laser 12 for removing a tissue 14 from a cornea of a human or animal eye 16 by means of photodisruption and/or photoablation. For example, the tissue 14 can represent a lenticule or also volume body, which can be separated from the cornea by the eye surgical laser 12 for correcting a visual disorder. A geometry of the tissue 14 to be removed can be provided by a control device 18, in particular in the form of control data, such that the laser 12 emits pulsed laser pulses in a pattern predefined by the control data into the cornea of the eye 16 to remove the tissue 14. Alternatively, the control device 18 can be a control device 18 external with respect to the treatment apparatus 10.

Furthermore, FIG. 1 shows that the laser beam 20 generated by the laser 12 can be deflected towards the eye 16 by means of a beam deflection device 22, namely a beam deflection apparatus such as for example a rotation scanner, to remove the tissue 14. The beam deflection device 22 can also be controlled by the control device 18 to remove the tissue 14.

Preferably, the illustrated laser 12 can be a photodisruptive and/or photoablative laser, which is formed to emit laser pulses in a wavelength range between 300 nanometers and 1400 nanometers, preferably between 700 nanometers and 1200 nanometers, at a respective pulse duration between 1 femtosecond and 1 nanosecond, preferably between 10 femtoseconds and 10 picoseconds, and a repetition frequency of greater than 10 kilohertz, preferably between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

By the laser 12 of the treatment apparatus 10, tissue 14 can also be removed for correcting presbyopia for the eye 16, in particular for providing a bi-aspherical or multi-aspherical refractive power distribution, in which different areas of refractive power distributions are for example provided for a near and far vision.

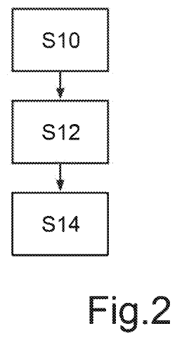

Up to now, the correction data for the respective eyes of a patient has been ascertained separately from each other without taking into account that a vision perception of the patient by both eyes is correlated. In order to take this into account and to provide control data with improved correction data for presbyopia correction, the method shown in FIG. 2 can be performed by the control device 18.

Now referring to FIG. 2, a schematic method diagram for providing control data for the ophthalmological laser 12 of the treatment apparatus 10 for correcting presbyopia is shown.

In a step S10, first correction data for a first eye 16 of a patient can be ascertained for correcting presbyopia. Visual disorder data of the patient can be previously determined in diagnostic measurements, from which the first correction data is determined. The correction data can include the refractive power or refractive power distributions, which are to be applied for the presbyopia correction of the first eye.

In a step S12, second correction data for the other second eye (not shown) of the patient can then be determined, wherein it is ascertained depending on the first correction data of the first eye 16. The first correction data can be multiplied and/or added by a patient-specific parameter, by which a correlation of the two eyes can be established. The patient-specific parameter can be preset based on social factors of the patient, in particular based on an age and/or a profession. Alternatively or additionally, the patient-specific parameter can also depend on a preoperative refraction and/or a residual accommodation of the respective eyes. For calculating the second correction data for the second eye, it can for example be provided that the ascertained first correction data is multiplied by the patient-specific parameter, wherein the patient-specific parameter has a value between −1 and 1. This means that for example at a value of 1, the same correction is performed at both eyes, and at a value of −1, the correction for the second eye is inverted. For example, a bi-aspherical refractive power distribution can be provided, wherein in the first eye 16, a central area is optimized for a near vision and an area adjoining thereto for a far vision. By the patient-specific parameter of −1, the central area can be optimized for a far vision and the area adjoining thereto for a near vision for the second eye. This approach can for example be performed for patients above a preset age threshold value. For the patient-specific parameter, intermediate ranges between −1 and 1 can also be selected to mitigate an effect for the second eye, wherein the value of 0 is preferably excluded, which means no treatment of the second eye. Alternatively or additionally, the patient-specific parameter can also be added to the first correction data to obtain the second correction data, wherein a diopter value in a range of values from −2 diopters to 2 diopters can preferably be used.

Furthermore, it can be provided that a centering of a correction profile, which is provided in the correction data, is differently preset in the respective eye. In particular, an offset of a pupil center to a corneal vertex can be present viewed in radial direction, which means that they are not on a common axis. In order to compensate for or consider the effects, which can arise by this offset, the correction of the first eye 16 can for example be planned at the level of the pupil center viewed in radial direction and the correction profile of the other eye at the level of the corneal vertex viewed in radial direction. Thus or by additional measurements, higher order aberrations can also be taken into account for planning the correction data of the respective eye.

Finally, control data for the ophthalmological laser 12 can be provided in a step S14, which comprises the first and second correction data for the respective eyes.

This bilateral correction is based on a model for extending the binocular focus depth within values, which do not compromise themselves. For example, a monocular vision can be restricted to less than 0.6 diopters or a target astigmatism is restricted to less than 1 diopter. Furthermore, a binocular fusion, for example a defocusing difference between the eyes, can be no more than 1.25 diopters to avoid an excessive difference between the eyes. The method can preferably be applied for different treatments, for example Lasik, PRK and lenticule extraction, in particular for hyperopia, myopia and treatments with and without astigmatism.

Overall, the examples show how an improved presbyopia correction can be provided by the invention.

1. A method for providing control data for an ophthalmological laser of a treatment apparatus for correcting presbyopia, wherein the method comprises the following steps performed by a control device:
ascertaining first correction data for an eye of a patient for correcting the presbyopia from predetermined visual disorder data;
ascertaining second correction data for another eye of the patient, wherein the second correction data is calculated by means of a calculation operation of the first correction data with a patient-specific parameter; and
providing the control data for the ophthalmological laser, which includes the first and second correction data for the respective eyes.

2. The method according to claim 1, wherein the first and/or the second correction data includes bi-aspheric, tri-aspheric and/or multi-aspheric refractive power distributions.

3. The method according to claim 1, wherein the calculation operation is a multiplication and the patient-specific parameter has a value in a range of values from −1 to 1.

4. The method according to claim 1, wherein the calculation operation is an addition and the patient-specific parameter has a diopter value in a range of values from −2 diopters to 2 diopters.

5. The method according to claim 1, wherein a center of the treatment is differently planned for each eye.

6. The method according to claim 1, wherein the patient-specific parameter is determined based on a preoperative refraction, a residual accommodation, an age and/or a profession.

7. The method according to claim 1, wherein higher order aberrations are also taken into account in the respective correction data.

8. A method for controlling a treatment apparatus, wherein the method comprises the following steps:
the method steps according to claim 1, and
transferring the provided control data to a respective ophthalmological laser of the treatment apparatus.

9. A control device, which is configured to perform the method according to claim 1.

10. A treatment apparatus with at least one ophthalmological laser for separation of a corneal volume with predefined interfaces of a human or animal eye by means of optical breakthrough, wherein the optical breakthrough comprises photodisruption and/or photoablation, and at least one control device according to claim 9.

11. (canceled)

12. A non-transitory computer-readable medium, on which a computer program is stored, the computer program including commands, which cause the treatment apparatus to execute the method according to claim 1.
